(19) United States
(12) Patent Application Publication
(10) Pub. No.: US 2009/0082761 A1
(43) Pub. Date: Mar. 26, 2009

(54) CRYOSURGICAL INSTRUMENT WITH EMBEDDED SEPARATING UNIT

(75) Inventors: Alexander LEVIN, Binyamina (IL); Didier TOUBIA, Raanana (IL); Miron KAGANOVICH, Haifa (IL)

Correspondence Address: HESLIN ROTHENBERG FARLEY & MESITI PC, 5 COLUMBIA CIRCLE, ALBANY, NY, 12203, US

(73) Assignee: ARBEL MEDICAL, LTD., Yokneam (IL)

(21) Appl. No.: 12/237,805

(22) Filed: Sep. 25, 2008

Related U.S. Application Data
(60) Provisional application No. 60/975,242, filed on Sep. 26, 2007.

(51) Int. Cl. A61B 18/02

(52) U.S. Cl. 606/22

(57) ABSTRACT

A cryosurgical instrument, in which a separator is installed in a central feeding lumen and/or in a cryotip to separate gaseous and liquid components of a cryogen.

### FIELD OF THE INVENTION

The present invention relates to the area of cryosurgical equipment, and, specifically, to cryoprobes or cryocatheters intended to be inserted in tissue or to be brought in contact with the tissue in order to perform cryosurgical treatment, which feature embedded separators for separating liquid and gaseous cryogen.

### BACKGROUND OF THE INVENTION

Cryoprobes which “boil” a liquid cryogen, when this liquid cryogen is supplied from an external source into the cryoprobe, are known for performing cryosurgical procedures. Generally, a cryogen is delivered into a cryoprobe in the form of mist, i.e., in the form of small droplets distributed in the vapors of the cryogen itself. This is caused by the fact that a certain fraction of the liquid cryogen is evaporated inevitably on the way to the cryoprobe as a result of imperfect thermal insulation of the delivery hose. The cryogen mist cannot be separated completely in the internal cavity of the cryotip (the distal section of the cryoprobe) between the liquid and gaseous phases without application of special measures. Without such special means, it is impossible to completely use the liquid fraction of the cryogen for effective freezing of a treated tissue.

There were some previous attempts to solve this problem; however all suffer from significant drawbacks, due to positioning of a separation unit outside the cryosurgical instrument.

U.S. Pat. No. 5,324,286 describes a cryogenic apparatus which comprises a coolant system and a probe having a cryogenically-cooled tip. The probe is formed from an elongated housing having a distal end closed by the tip and a proximal end connected to the coolant system. The housing is adapted to receive cryogen droplets entrained in a warm carrier gas stream supplied by the coolant system. The carrier gas stream passes through the housing such that the entrained cryogen droplets are transported to the distal end of the probe for cooling the tip. The tip is cryogenically cooled by the cryogen droplets which are collected at the base of the tip. More specifically, the carrier gas transports the entrained cryogen droplets, through the inlet tube to the distal end of the probe where, because of their inertia, the droplets cannot follow the 180 degree bend of the returning carrier gas stream. Instead, the droplets are deposited and stored in a porous heat sink positioned in the tip. The porous heat sink is positioned such that it is in thermal contact with a tip head. Both the porous heat sink and the tip head are formed of a thermally conductive material. The liquid deposited in the heat sink from impinging droplets is evaporated by heat supplied by the object to be cooled, such as tumor tissue which is placed in contact with the tip head. Accordingly, the tip reaches temperatures commensurate with the saturation temperature of the evaporating liquid cryogen.

U.S. Pat. No. 5,254,116 describes a cryoprobe with a separation means in the form of a liquid nitrogen supply tube, which is provided with a plurality of small vent holes to vent gas formed or present in the refrigerant supply tube to the return refrigerant flow channel. The vent holes also allow a small amount of liquid nitrogen to vent into the return flow channel to further reduce the temperature differential between the sub-cooled liquid nitrogen supply and the counter-current flowing return refrigerant.

An analogous technical solution is described in U.S. Pat. No. 5,520,682. However, such a design for a separator cannot ensure effective separation of liquid and gaseous phases of the cryogen mixture.

An article by S. L. Qi et al., “DEVELOPMENT AND PERFORMANCE TEST OF A CRYOPROBE WITH HEAT TRANSFER ENHANCEMENT CONFIGURATION”, CRYOGENICS, Vol 46 (2006), pages 881-887, describes a cryosurgical system, in which liquid nitrogen is supplied into a cryoprobe from a dewar flask. In order to improve quality of the liquid-gaseous mixture supplied from the dewar flask, there is a separator, which is positioned immediately after the dewar flask and serves for separation between the liquid and gaseous phases of the stream.

However, this technical solution cannot provide complete separation of gaseous and liquid phases due to additional gasification of the liquid nitrogen, which occurs in the supplying hose of the system and in the cryoprobe itself as a result of imperfections of their respective thermal insulations.

A detailed analysis of different industrial systems of droplet separation is presented in the book: “Droplet separation”, by Armin Burkholz, VCH, New York, 1990, 229 pp. However, this book does not describe any useful solutions for cryosurgical instrument separators.

### SUMMARY OF THE INVENTION

The background art does not teach or suggest a solution that solves the problem of separation of gaseous and liquid phases of a liquid cryogen, due to partial evaporation of the cryogen as it is delivered to, and boils within, a cryoprobe.

The present invention overcomes the above problems of the background by separating liquid droplets of the cryogen and its gaseous phase immediately near the internal surface of the cryotip and directing the obtained liquid phase on the internal surface with following its boiling and evaporation in order to solve the above problem.

The present invention relates to a number of illustrative embodiments of a cryoprobe separator, which is situated near or in a central feeding lumen of the cryoprobe and which, among many advantages, solves the above technical problem.

In an embodiment of a cryoprobe according to the present invention, a separator is installed in its central feeding lumen, for separating gaseous and liquid components of the cryogen. The separator preferably features one or more inserts which increase the internal surface area and hence increase the separating capabilities of the separator.

The separator, in some embodiments, is optionally and preferably constructed as a metal strip with a suitable width to be introduced into the distal section of the central feeding lumen. The proximal section of the metal strip is preferably provided with dimples or other surface indentations directed alternatively downwards and upwards, thereby enabling this metal strip to function as a wave-plate separator. In addition, the distal section of the strip, which is situated outside the central feeding lumen, provides a liquid cryogen film immediately on the bottom surface of the cryotip, due to adhesion of the liquid cryogen film to the surface of the strip.

Various implementations may optionally be used to install the metal strip in the central feeding lumen. For example, the distal section of the metal strip may optionally be of greater width than its proximal section. The greater width of this distal section thereby maintains the position of the metal strip with regard to the internal wall of the cryotip.

In another optional embodiment, the distal section of the central feeding lumen is preferably provided with some slits, which lock the edges of the metal strip.

In addition, the distal section of the central feeding lumen may optionally be provided with a plurality of wire spirals, in which the distal sections of these spirals are situated outside the distal section of the central feeding lumen. A bushing with a toothed distal section is preferably installed on the distal section of the central feeding lumen to hold these wire spirals.

Separating members may optionally and preferably be placed as well in the gap between the distal section of the central feeding lumen and the external wall of the cryotip.

The separating members of the present invention may optionally be provided in various implementations, according to different embodiments of the present invention.

These separating members may optionally, for example, feature a plurality of spiral wires or other thin spiral structures situated in the aforementioned gap between the distal section of the central feeding lumen and the external wall of the cryotip.

In another embodiment, a wire spiral is preferably wrapped around the distal section of the central feeding lumen.

Alternatively, the wire spirals are first soldered or otherwise attached to a thin metal strip, which is bent afterwards and installed on the internal wall of the cryotip by soldering or gluing, or any other appropriate attachment method.

In another embodiment, the separating members preferably feature a plurality of arched corrugated plates arranged in the gap between the central feeding lumen and the internal wall of the cryotip.

In yet another embodiment, separating members preferably feature a plurality of flat rings with teeth directed inwards and some tenons on their external edges; these tenons hold the position of the flat rings on the internal wall of the cryotip with good thermal contact between the internal wall of the cryotip and these tenons.

The flat rings are preferably fabricated from a soft metal with high thermal conductivity.

Alternatively, a plurality of flat rings with greater thickness is used; preferably the rings are fabricated from metal which more preferably has high thermal conductivity. Each flat metal ring is provided with a circular groove and some perforations. The diameter of the internal central opening of the flat ring ensures its tight fit on the central feeding lumen. On the other hand, the outer diameter of the flat ring ensures its tight fit on the internal wall of the cryotip.

In addition, each pair of neighboring flat rings can be mutually oriented to intercept droplets that fall within a first ring of the pair of rings by the second ring of the pair of rings. In addition, the flat metal rings serve as thermal bridges between the central feeding lumen and the internal wall of the cryotip, and their surfaces enhance the total surface of internal heat exchange with the cryogen.

In yet another embodiment, a separating member features a bushing which is divided into a plurality of sections by necks.

The outer diameter of the bushing fits the internal diameter of the cryotip, and its internal diameter fits the outer diameter of the distal section of the central feeding lumen, thereby tightly fitting the bushing on the distal section of the central feeding lumen.

The outer surfaces of the sections of the bushing are provided with multiple threads, with neighboring sections having multiple threads of opposite directions. This ensures separation of the cryogen droplets from its gaseous phase, and, on the other hand, the sections of the bushing are divided by necks (the sections with reduced diameter). The sections of the bushing with the multiple threads provide thermal bridges between the central feeding lumen and the cryotip.

In another embodiment, there is an electret coating on one or more parts of the internal surface of the cryotip. The “electret coating” is made from any type of electrified material as is known in the art. The electrostatic field created by this electret coating causes precipitation of cryogen droplets and, in such a way, separation of the gaseous and liquid phases of the cryogen. It should be noted that this method of separation may optionally be combined with the mechanical methods described above.

In another embodiment, separating cryogen droplets and cryogen gas is performed through induction of a vortex. In this case, the distal section of the central feeding lumen is provided with a bushing; the distal end of this bushing is sealed, and the bushing is provided with a set of longitudinal slots, which cause vortex flow in the gap between the central feeding lumen and the internal wall of the cryotip.

In yet another embodiment, the separation of cryogen droplets and cryogen gas is at least partially accomplished through the provision of packing material, which is installed in the internal cavity of cryotip on the distal section of the central feeding lumen. This packing material is preferably gear-shaped and fabricated from a micro-mesh metal screen (for example and without any intention of being limiting, from micro-mesh copper screens manufactured by Industrial Netting Co., Minneapolis, Minn. USA). The micro-mesh metal screen is then preferably bent to the preferred shape.

### BRIEF DESCRIPTION OF THE DRAWINGS

FIG. 1a is an axial cross-section of the cryoprobe with a strip inserted partially into the central feeding lumen.

FIG. 1b and FIG. 1c are a frontal view and a longitudinal cross-section, respectively, of the metal strip of FIG. 1a.

FIG. 2a is an axial cross-section of the cryotip with a set of flat metal rings with toothed internal edges.

FIG. 2b is a frontal view of the flat metal ring of FIG. 2a.

FIG. 3a is an axial cross-section of the cryoprobe with a separating member in the form of a metal strip with a set of soldered metal spirals.

FIG. 3b and FIG. 3c are a cross-section of the strip with the soldered metal spirals and a transverse cross-section of the cryotip, respectively.

FIG. 4 is an axial cross-section of the cryoprobe with a metal spiral wound on the central feeding lumen.

FIG. 5a is an axial cross-section of the cryoprobe with a set of perforated metal rings.

FIG. 5b and FIG. 5c are a top view and a radial cross-section, respectively, of the perforated metal ring.

FIG. 6a is an axial cross-section of the cryoprobe with the separating members, as arc shaped corrugated plates arranged in the gap between the central feeding lumen and the internal wall of the cryotip.

FIG. 6b is a radial cross-section and a transverse cross-section of the cryotip with arced corrugated plates arranged in the gap between the central feeding lumen and the internal wall of said cryotip.

FIG. 7a is an axial cross-section of the cryoprobe with the separating member in the form of a multiple neck bushing provided with multiple threads.

FIG. 7b and FIG. 7c are a side view and a view from the face plane, respectively, of the bushing of FIG. 7a.

FIG. 8 is an axial cross-section of the cryoprobe with an electret coating of the distal section of the central feeding lumen.

FIG. 9a is an axial cross-section of the cryoprobe with a separation unit designed as a bushing with tangential slots.

FIG. 9b is a transverse cross-section A-A of the cryotip of the probe of FIG. 9a.

FIG. 10a is an axial cross-section of the distal section of an exemplary embodiment of a cryoprobe with a separation unit in the form of packing material constructed from a micro-mesh metal screen.

FIG. 10b is a transverse cross-section A-A of the cryotip of the probe of FIG. 10a.

### DESCRIPTION OF PREFERRED EMBODIMENTS

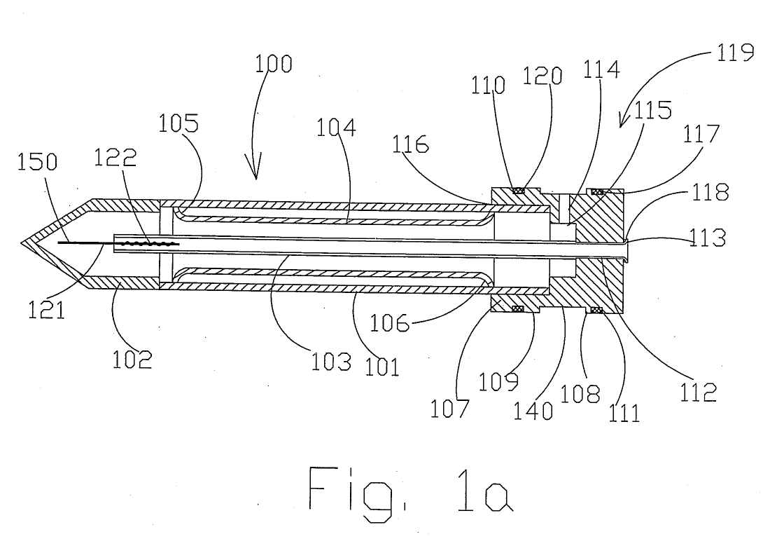

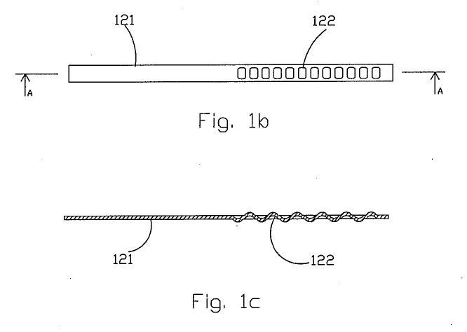

FIG. 1a is an axial cross-section of a first embodiment of the cryoprobe with a strip inserted partially into the central feeding lumen.

This embodiment of cryoprobe 100 according to the present invention comprises shaft 101, which ends at its distal edge with cryotip 102. Shaft 101 is preferably fabricated from a rigid material.

A central feeding lumen 103 is situated at least partially in shaft 101 such that the proximal end of the central feeding lumen 103 somewhat protrudes from the proximal end of shaft 101. Cryogen enters central feeding lumen 103 through an opening 113. The proximal sections of shaft 101 and the central feeding lumen 103 preferably serve for installation of a male unit 119 for a quick coupling mechanism.

Thermal insulation of shaft 101 is ensured by an intermediate tube 104 with two flanged ends 105 and 106, wherein the outer diameter of the formed flanges 105 and 106 conforms to the internal diameter of the shaft 101, and is preferably less than the latter diameter. Friction between the internal surface of shaft 101 and flanged ends 105 and 106 ensures stable positioning of the intermediate tube 104 within shaft 101.

The male unit 119 of the quick coupling mechanism, which is installed on the proximal sections of shaft 101 and the central feeding lumen 103, preferably comprises bushing 107, in which the outer and internal surface of this bushing 107 are divided into a plurality of sections with different diameters.

The outer surface of bushing 107 comprises proximal, middle and distal cylindrical sections 108 and 140, 109 respectively; the outer diameter of the distal section 109 is preferably somewhat larger than the outer diameter of the proximal section 108. The proximal and distal cylindrical sections 108 and 109 are provided with annular grooves 111 and 110 for installation of O-rings 117 and 120 respectively, preferably constructed from a cryogenically stable polymer.

The inner surface of bushing 107 is also preferably shaped in a stepped form: it has distal, intermediate and proximal sections 116, 115 and 112 respectively with progressively decreasing diameters.

Bushing 107 is installed on the proximal sections of shaft 101 and the central feeding lumen 103 in such a manner that the distal section of the inner surface of the bushing is fitted tightly on the proximal section of the shaft 101 and the proximal inner surface 112 of bushing 107 is fitted slidingly on the turned-along proximal section of the central feeding lumen 103 with decreased diameter. After positioning bushing 107 on the proximal section of shaft 101, the proximal edge of the central feeding lumen 103 is flanged, to which is applied a deformable O-ring 118, preferably constructed from a cryogenically stable polymer, for sealing the gap between the proximal sections of the internal surface of bushing 107 and the central feeding lumen 103. A channel 114 communicates between the internal and external spaces of the bushing in the place of its inner intermediate section 115 and its outer proximal section 108.

As shown in FIGS. 1B and 1C, a metal strip 121 is partially arranged in the distal section of the central feeding lumen 103, and at least the proximal section of the metal strip 121 is provided with dimples or other indentations 122, directed alternatively downwards and upwards, thereby enabling this metal strip 121 to function as a wave-plate separator. In addition, the distal section 150 of the strip 121, which is situated outside the central feeding lumen 103, provides a liquid cryogen film immediately on the bottom surface of the cryotip 102, due to adhesion of the liquid cryogen film to the surface of the strip 121.

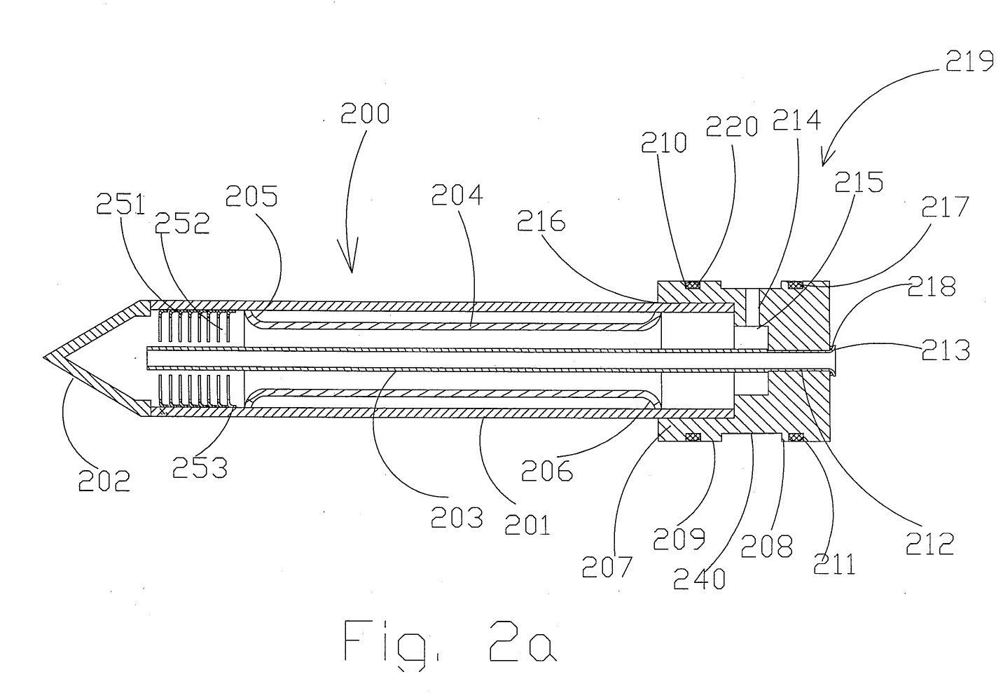

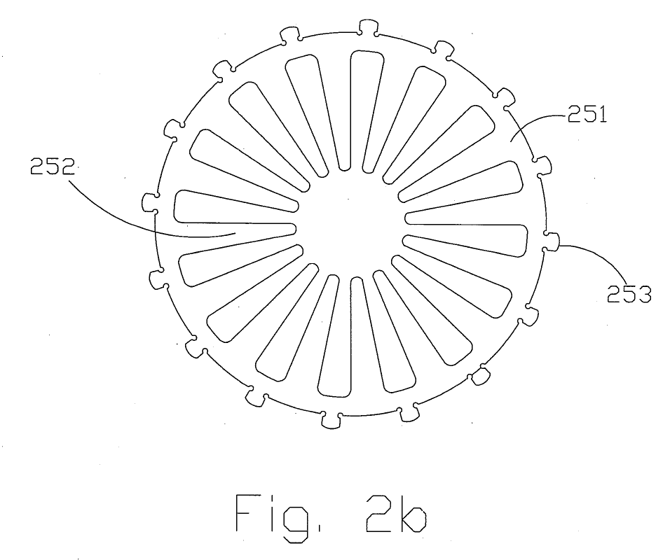

FIG. 2a is an axial cross-section of the cryotip with a set of flat metal rings with toothed internal edges and FIG. 2b is a frontal view of a flat metal ring of FIG. 2a. This embodiment of cryoprobe 200 comprises shaft 201, which ends at its distal edge with cryotip 202. Shaft 201 is preferably fabricated from a rigid material. Elements with the same number plus “100” have the same or similar function as the corresponding elements in FIG. 1.

A plurality of flat metal rings 251 with toothed internal edges 252 is arranged in the internal space of cryotip 202. The external edges of the flat metal rings 251 are provided with tenons 253, which are bent transversally to the planes of the flat metal rings 251 and provide good thermal contact with the internal wall of cryotip 202.

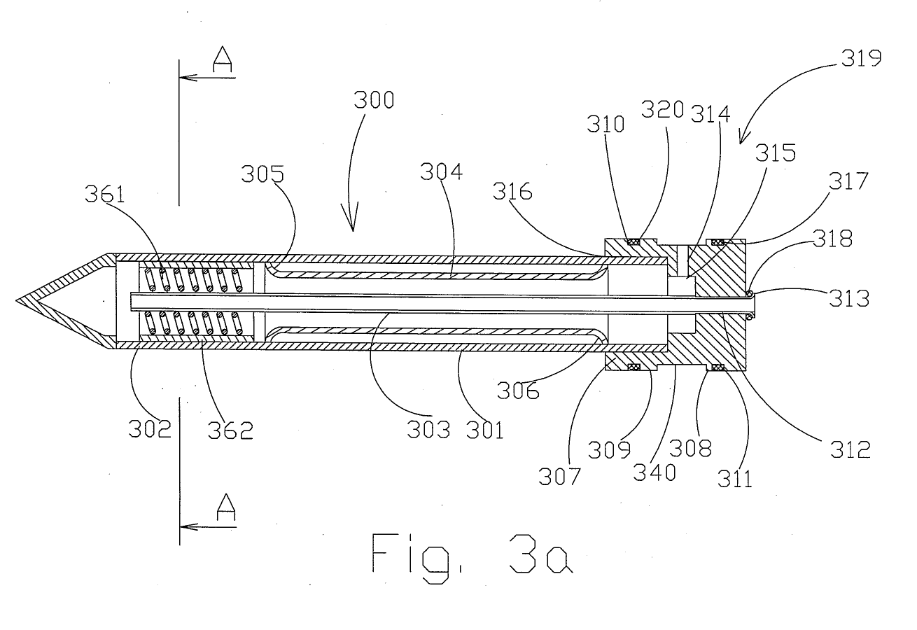

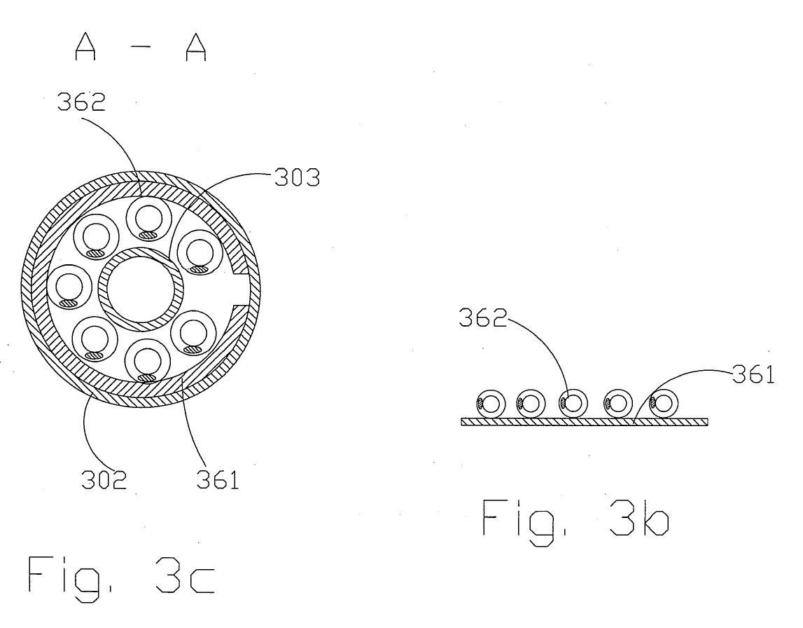

FIG. 3a is an axial cross-section of another embodiment of a cryoprobe according to the present invention, with a separating member in the form of a metal strip with a set of soldered or otherwise attached metal spirals.

Elements with the same number plus “200” have the same or similar function as the corresponding elements in FIG. 1.

The internal space of cryotip 302 is preferably provided with a plurality of wire spirals 362, which are soldered or otherwise attached to a thin metal strip 361; this thin metal strip 361 is preferably bent afterwards and installed on the internal wall of cryotip 302 by soldering or gluing.

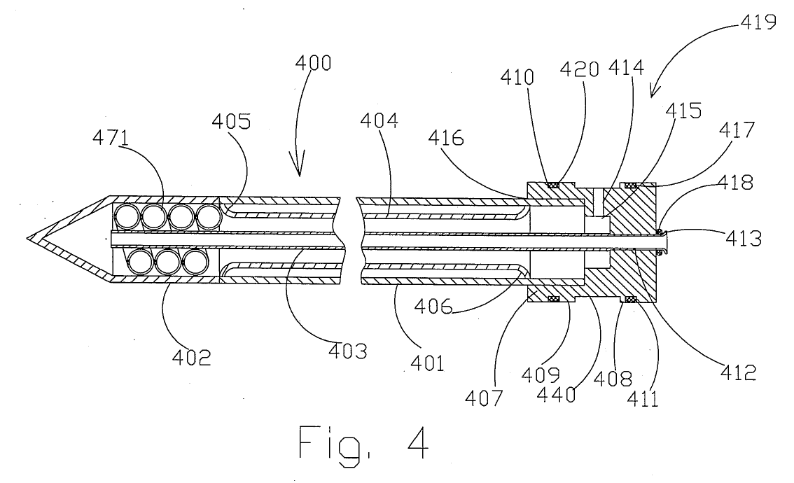

FIG. 4 is an axial cross-section of another embodiment of a cryoprobe according to the present invention, with a metal spiral 471 wound on the central feeding lumen, which serves as a separator. Elements with the same number plus “300” have the same or similar function as the corresponding elements in FIG. 1.

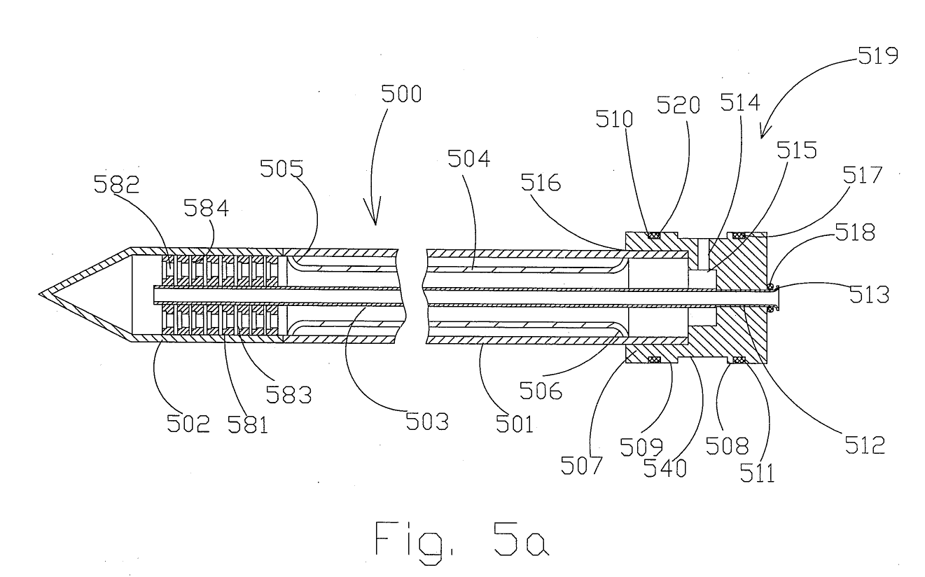

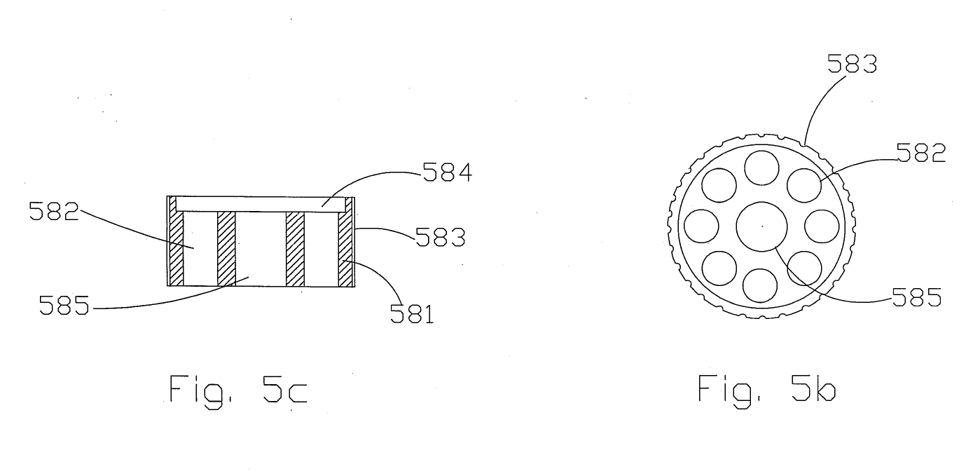

FIG. 5a is an axial cross-section of another embodiment of a cryoprobe according to the present invention, with a plurality of perforated metal rings. Elements with the same number plus “400” have the same or similar function as the corresponding elements in FIG. 1.

A plurality of metal perforated rings 581 is installed on the distal section of the central feeding lumen 503. Perforations 582 allow passage of the cryogen mist with separation of the liquid cryogen component from the gaseous cryogen component. In another version, the external surface of the metal perforated rings 581 is provided with ribs 583, which improves contact of the liquid cryogen component with the internal wall of cryotip 502.

FIG. 5b and FIG. 5c show a top view and an axial cross-section, respectively, of the metal perforated ring 581.

The metal perforated ring 581 comprises perforations 582, a central opening 585, a circular recess 584 and ribs 583.

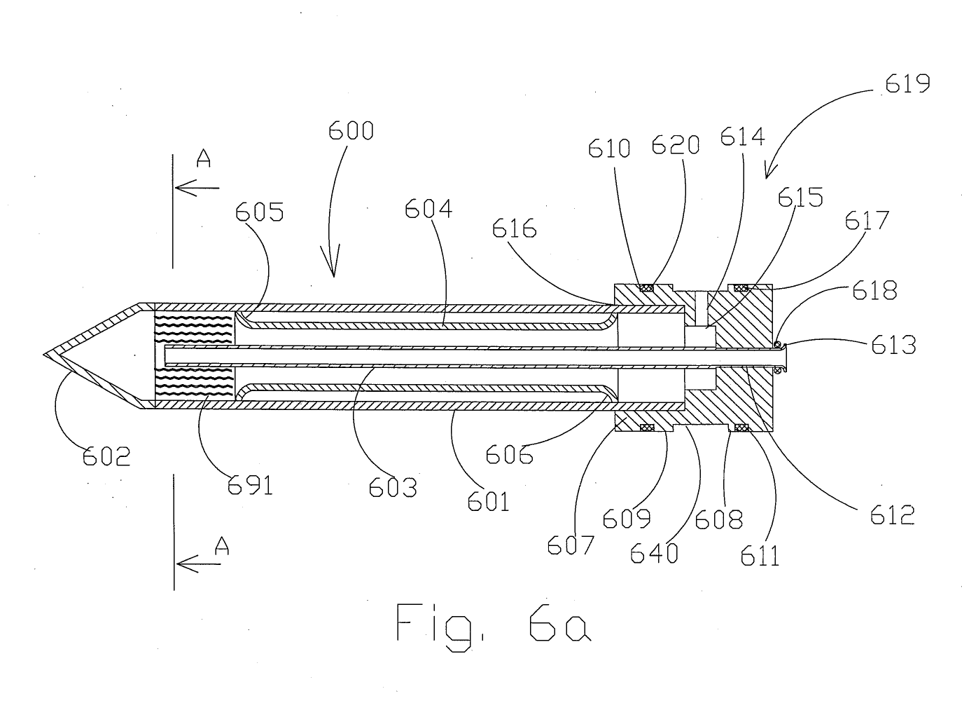

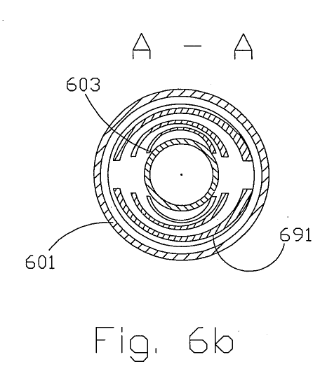

FIGS. 6a and 6b show another embodiment of a cryoprobe according to the present invention, with a plurality of separating members, which feature arc shaped corrugated plates arranged in the gap between the central feeding lumen and the internal wall of the cryotip. Elements with the same number plus “500” have the same or similar function as the corresponding elements in FIG. 1.

Separating members 691, which are constructed as arc shaped corrugated plates, are arranged in the gap between the central feeding lumen 602 and the internal wall of cryotip 602.

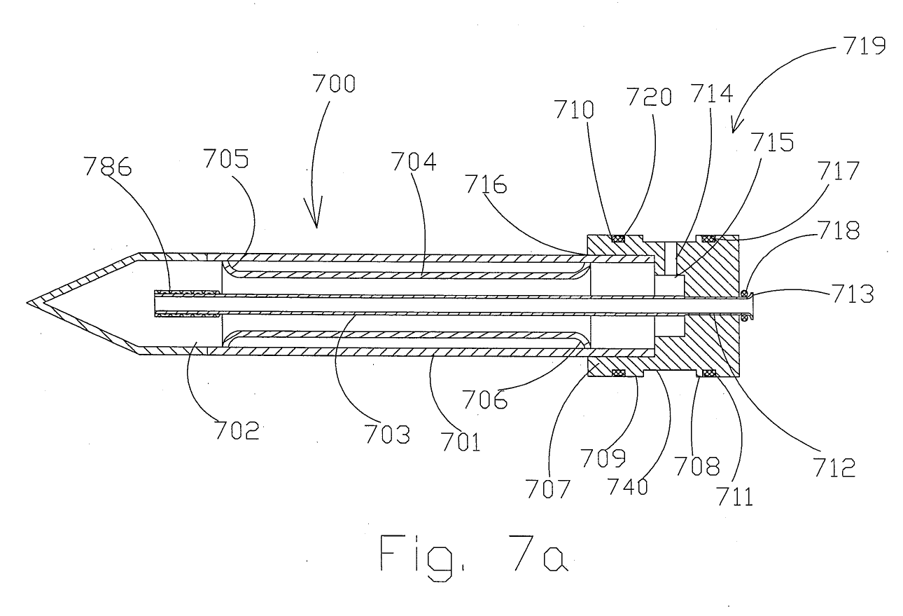

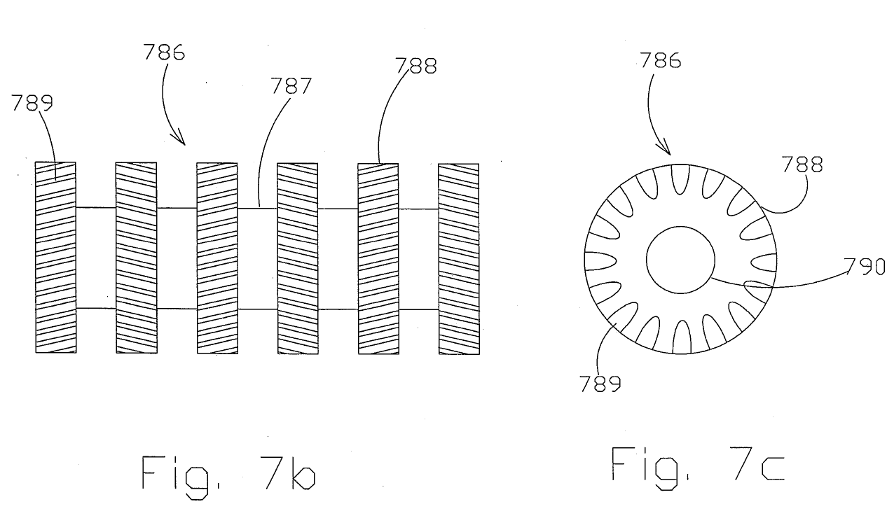

FIG. 7a is an axial cross-section of another embodiment of a cryoprobe according to the present invention, with the separating member in the form of a bushing provided with multiple threads and having multiple necks. Elements with the same number plus “600” have the same or similar function as the corresponding elements in FIG. 1.

FIG. 7b and FIG. 7b show an axial cross-section and a top view of the bushing, respectively.

A bushing 786 features a plurality of necks 787 that divide it into a plurality of sections 788.

The outer surfaces of the different sections 788 of bushing 786 are provided with multiple threads 789, such that the neighboring sections have threads of opposite directions. Bushing 786 features a central opening 790.

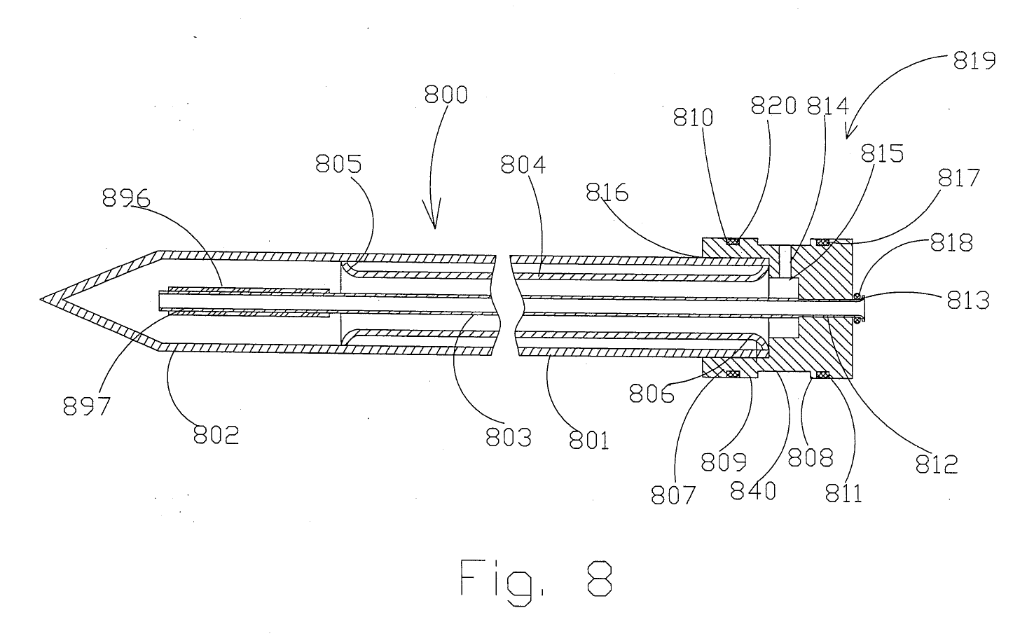

FIG. 8 is an axial cross-section of another embodiment cryoprobe according to the present invention, with an electret coating of the distal section of the central feeding lumen. Elements with the same number plus “700” have the same or similar function as the corresponding elements in FIG. 1.

An electret coating 896 on the outer surface of the distal section 897 of the central feeding lumen 803 ensures separation of the cryogen mist, due to the electrostatic field generated between this electret coating 896 and the internal wall of cryotip 802.

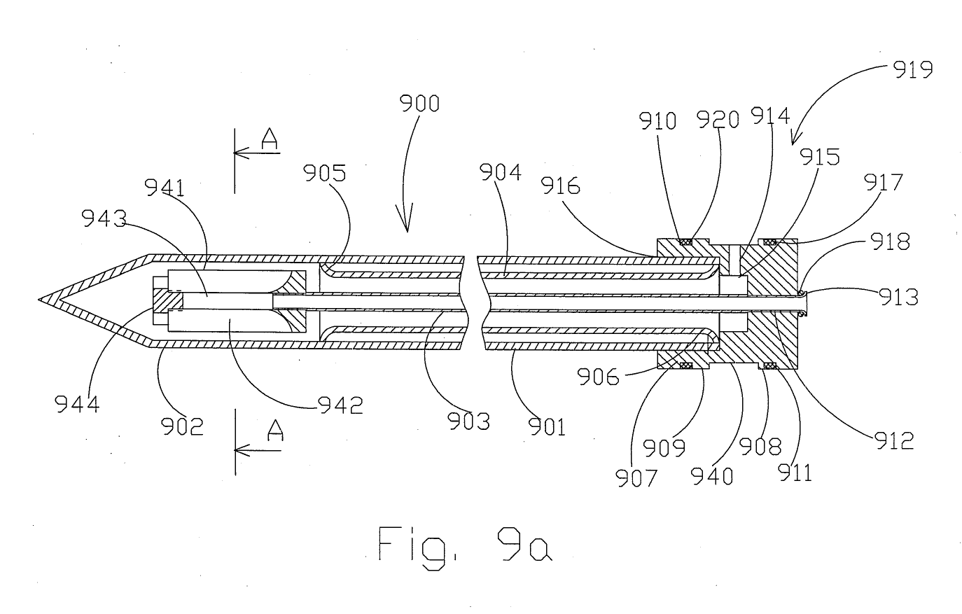

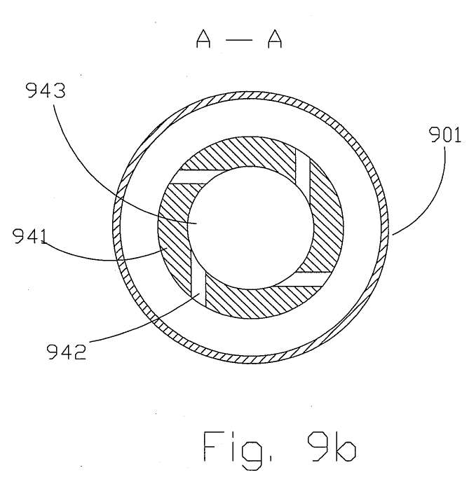

FIG. 9a is an axial cross-section of another embodiment of a cryoprobe with a separation unit designed as a bushing with tangential slots, and FIG. 9b is a transversal cross-section A-A of the cryotip of the probe of FIG. 9a. Elements with the same number plus “800” have the same or similar function as the corresponding elements in FIG. 1.

A distal bushing 941 is installed on the distal section of the central feeding lumen 903. The distal end of the internal hole 943 of the distal bushing 941 is sealed by plug 944, and longitudinal slots 942 are provided in the distal bushing 941, which ensure vortex flow of the cryogen mixture in the gap between the distal bushing 941 and the internal wall of cryotip 902.

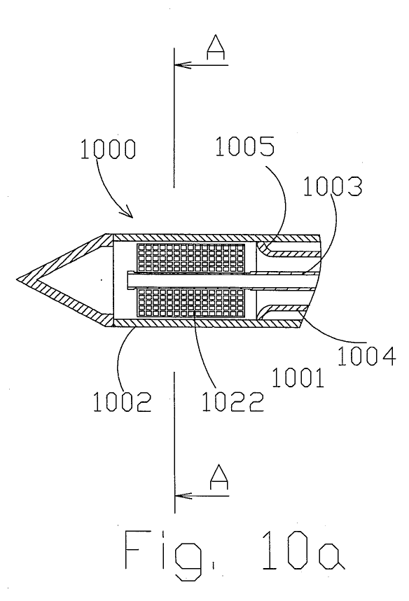

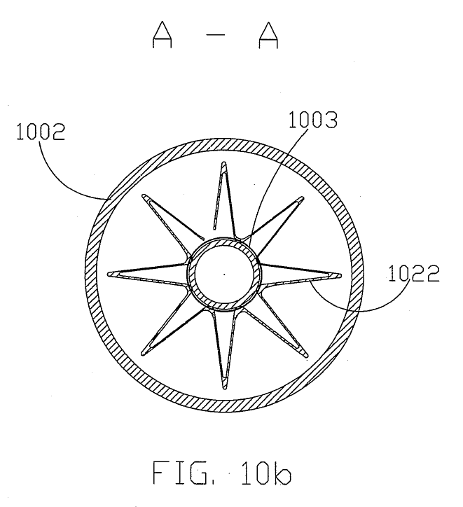

FIG. 10a is an axial cross-section of the distal section of the cryoprobe with a separation unit that includes packing material constructed from a micro-mesh metal screen as a non-limiting example. The packing material is preferably in the form of a gear fabricated by bending a rectangular piece of the micro-mesh metal screen.

FIG. 10b is a transverse cross-section A-A of the cryotip of the cryoprobe of FIG. 10a.

Together, FIG. 10a and FIG. 10b show a portion of a cryoprobe 1000, with a distal section of a cryoprobe shaft 1001 featuring an intermediate tube 1004 and its flanged end 1005. A central feeding lumen 1003 for receiving the cryogen terminates in the internal cavity of cryotip 1002. There is a gear-shaped packing structure 1022, which is optionally and preferably fabricated from a micro-mesh metal screen (for example, from micro-mesh copper screens manufactured by Industrial Netting Co., Minneapolis, Minn. USA), which is installed on the distal section of the central feeding lumen 1003 for separating the gaseous and liquid portions of the cryogen.

Persons skilled in the art will appreciate that the present invention is not limited to what has been particularly shown and described hereinabove. Rather the scope of the present invention is defined by the appended claims and includes both combinations and sub combinations of the various features described hereinabove as well as variations and modifications thereof, which would occur to persons skilled in the art upon reading the foregoing description.

What is claimed is:

1. A cryosurgical instrument operating with a supplied gaseous-liquid mixture of a cryogen, said cryosurgical instrument comprising:
an external shaft;
a cryotip joined with a distal edge of said external shaft; and
a separation unit for separating of a gaseous component of the cryogen from a liquid component of the cryogen, being situated at least partially in an internal space of said cryotip.

2. The cryosurgical instrument of claim 1, further comprising:
a central feeding lumen, wherein a majority of said lumen is situated in an internal space of said external shaft and cryotip and a proximal edge of said external shaft is joined sealingly with a proximal section of said central feeding lumen;
a proximal coupling unit, which includes an inlet connection for delivery of said gaseous-liquid mixture of said cryogen and an outlet connection for removal of a gaseous component of said cryogen; and
a thermal insulation unit of said external shaft.

3. The cryosurgical instrument of claim 1, wherein the separation unit comprises a metal strip partially arranged in a distal section of the central feeding lumen, and at least a proximal section of said metal strip is provided with dimples.

4. The cryosurgical instrument of claim 1, wherein the separation unit comprises a set of flat metal rings with toothed internal edges; said flat metal rings are arranged in the internal space of the cryotip; external edges of said flat metal rings are provided with tenons, and the tenons are bent transversally to planes of the flat metal rings and provide good thermal contact with an internal wall of the cryotip.

5. The cryosurgical instrument of claim 1, wherein the separation unit comprises a set of metal perforated rings installed on a distal section of the central feeding lumen.

6. The cryosurgical instrument of claim 5, wherein an external surface of each metal perforated ring is provided with ribs.

7. The cryosurgical instrument of claim 1, wherein the separation unit comprises wire spirals soldered to a thin metal strip; and said thin metal strip with said soldered wire spirals is bent and installed on an internal wall of the cryotip by soldering or gluing.

8. The cryosurgical instrument of claim 1, wherein the separation unit comprises a metal spiral wound on a distal section of the central feeding lumen.

9. The cryosurgical instrument of claim 1, wherein the separation unit comprises arc shaped corrugated plates arranged in a gap between the central feeding lumen and an internal wall of the cryotip.

10. The cryosurgical instrument of claim 1, wherein the separation unit comprises an electret coating of internal parts of the cryotip.

11. The cryosurgical instrument of claim 10, wherein an outer surface of the distal section of the central feeding lumen is provided with a layer of the electret coating.

12. The cryosurgical instrument of claim 1, wherein a distal bushing is installed on a distal section of the central feeding lumen; a distal end of an internal hole of said distal bushing is sealed by a plug, and longitudinal slots in said distal bushing ensure vortex flow of the cryogen mixture in a gap between said distal bushing and an internal wall of the cryotip.

13. The cryosurgical instrument of claim 1, wherein the separation unit comprises a gear-shaped structure of a micro-mesh metal screen.
